(19) United States
(12) Patent Application Publication
(10) Pub. No.: US 2008/0311100 A1
(43) Pub. Date: Dec. 18, 2008

(54) Treatment of Keratinous Dryness With Glycerides

(75) Inventors: Patricia Manissier, Levallois-Perret (FR); Christiane Montastier, Paris (FR); Antoine Piccirilli, Versailles (FR)

Correspondence Address: OLIFF & BERRIDGE, PLC, P.O. BOX 320850, ALEXANDRIA, VA, 22320-4850, US

(73) Assignee: Laboratoires Inneov, Asnieres (FR)

(21) Appl. No.: 11/920,259

(22) PCT Filed: May 15, 2006
(86) PCT No.: PCT/FR2006/050445

§ 371 Date: Feb. 14, 2008

Related U.S. Application Data
(60) Provisional application No. 60/754,214, filed on Dec. 28, 2005.

(30) Foreign Application Priority Data
May 16, 2005 (FR) 0551264

(51) Int. Cl. A61K 31/122; A61K 31/202; A61K 31/385; A61P 17/08; A61K 31/715; A61K 38/02

(52) U.S. Cl. 424/94.1; 514/460; 514/2; 514/54

(57) ABSTRACT

The present invention provides the use of medium chain glyceride(s) as an active ingredient for the preparation of a composition for oral and/or parenteral absorption and intended to prevent and/or treat dry and/or delicate keratinous substances.

The present invention relates primarily to the use of medium chain glycerides for the preparation of a composition for oral and/or parenteral absorption, intended to prevent and/or treat dryness of keratinous substances, in particular cutaneous dryness and especially for the treatment of disorders associated with dry and/or hypo-seborrheic skin.

It should be recalled that skin is constituted by three superposed layers: the epidermis on the surface, then the dermis, and then the deeper hypodermis, and further that it includes auxiliary structures such as sebaceous glands in particular.

The skin essentially acts as a barrier to the external medium, as a result of a complex organization and many factors. That function is based in particular on the quality of the epidermis, which depends in particular on the degree of hydrophobicity of the surface of the stratum corneum and the equilibrium between proliferation and differentiation of epidermal keratinocytes.

A break in cutaneous equilibrium can manifest itself in various manners. It can in particular result in triggering inflammatory processes, deregulating the sebaceous function, hyperkeratinization and an increase in insensible water loss and more generally, it can lead to cutaneous dryness. These events have a negative effect on skin comfort and/or cutaneous esthetic. They are also susceptible of affecting the health of the epidermis.

Thus, an alteration in the barrier function encourages abnormal penetration of pathogenic agents into the corneal layer and induces an increased release of pro-inflammatory substances at the origin of cutaneous inflammatory disorders that cause itching and tightness, two characteristic symptoms of dry skin.

A first alternative treatment for dry skin characterized by a deficiency in the constituent lipids of the barrier and/or hydrolipid film is aimed at topical administration of substances intended to restore the cutaneous barrier. Such substances are generally moisturizing agents capable of binding water, film-forming agents intended to retain water, agents capable of reconstructing the cutaneous barrier such as exogenous lipids that are constituents of intercellular cement and sebum, such as squalene, ceramides, fatty acids, or active ingredients like vitamin C capable of stimulating the endogenous synthesis of epidermal lipids.

However, that mode of administration implies frequent renewal of the topical applications, and has an efficacy limited to the zone benefiting from topical application, and can also generate unwanted secondary cutaneous effects.

Another alternative consists in providing an affected subject with generally oral supplements of fatty acids, in particular polyunsaturated fatty acids called PUFA of the ω3 and ω6 family types. In the skin, such fatty acids undergo biotransformations under the action of elongase and desaturase type enzymes to produce pro- or anti-inflammatory higher polyenes such as eicosapentoic acid EPA and docosahexenoic acid DHA, prostaglandins, leucotrienes. Thus it has been shown that in the atopic child, a supplement of grape seed oil, which is rich in stearidonic acid and gamma linolenic acid, two prostaglandin precursors, could correct inflammatory cutaneous disorders (F. Balli et al. Riv. Ital. Pediatr. (IJP) 1992; 18:639-643). Similarly, consumption of 2400 mg/day of blackcurrant seed oil, which is also rich in gamma linolenic and stearidonic acids, has proved active in the treatment of xerosis or dry skin, in particular in an older subject (Wade, The Nutrition & Dietary Consultant, September 1986, pages 4-17).

As indicated above, dry skin may also be the consequence of and/or associated with an endogenous insufficiency in the production of sebum by the sebaceous glands.

Along with sweat, sebum constitutes a natural moisturizer for the epidermis and can increase its suppleness and strength. It is essentially composed of a mixture of lipids of varying complexity. Conventionally, the sebaceous gland produces squalene, triglycerides, aliphatic waxes, cholesterol waxes, and possibly free cholesterol. The action of bacterial lipases converts a variable portion of the triglycerides formed into free fatty acids.

Conventionally, an amount of sebum of less than 100 μg/cm², measured at the facial T zone using the method described in French patent FR-A-2 368 708, may be considered to be characteristic of a hypo-seborrheic dry skin.

An example of hypo-seborrheic dry skin, or of skin that is becoming like that, is observed as skin ages. Thus, very often, in older subjects, in particular those over 50 years of age, xerosis is observed associated with a sebum deficiency. Further, insufficient sebum production may be induced by certain pharmaceutical treatments such as those involving corticoids.

The use, especially topical or oral use, of amides or esters of sugar and of fatty acids and in particular linolenic acid is also proposed in International document WO-A-04/034958 for the treatment of cutaneous dryness and in particular hypo-seborrheic dry skin.

The present invention aims to propose the use of other compounds that have unexpectedly been shown to be effective in the prevention and/or treatment in general of disorders associated with the dryness of keratinous substances.

In the context of the present invention, the term “keratinous substance” means the skin, scalp, mucosa, nails, and keratinous fibers of human or animal origin.

More precisely, the inventors have shown that medium chain glycerides, i.e. composed of fatty acids with a hydrocarbon chain containing 6 to 12 carbon atoms, in particular medium chain triglycerides, have proved effective in the treatment of dry and/or delicate keratinous substances. Unexpectedly, their oral administration can re-launch sebum production.

Medium chain glycerides are compounds deriving from the esterification of glycerol by one, two, or three molecules of medium chain fatty acids.

Medium chain triglycerides, also generally termed MCT, are composed of three molecules of medium chain fatty acid esterified by one molecule of glycerol. They are odorless, tasteless, clear substances that have a low viscosity.

Medium chain triglycerides are already known to be fast and direct sources of energetic supply, in particular due to their unique metabolic profile. Indeed, compared with long chain fatty acid triglycerides, after ingestion, medium chain triglycerides liberate short fatty acids that are far less hydrophobic than long chain fatty acids and that reach the liver directly via the portal vein without being previously incorporated into transporters called lipoproteins.

Thus, European patent EP-A-1 023 843 proposes a formulation for nutritional supplements that are particularly intended for people undertaking active sports.

MCTs have also been proposed as a source of fatty substances in medical nutritional products (United States patent application US 2004/0151757 or U.S. Pat. No. 5,223,285). In particular, their good digestibility because of their hydrolysis in part by lingual and gastric lipases, and their rapid metabolism makes their use an effective means for supplying energy in malabsorption situations (U.S. Pat. No. 6,194,379). They have also been proposed for nutritional formulations with reduced calories and/or fat content more particularly intended for overweight people (US 2005/0143459).

Further, those compounds have also been proposed for topical dermatological use (DE 198 57 491, CA 1 253 807, EP-A-1 214 930 or JP 2005-104928).

Unexpectedly, the inventors have shown that such compounds have also proved to be particularly effective when administered in a non topical manner, in particular orally and/or parenterally, to treat disorders associated with dry or delicate keratinous substances, in particular dry skin, and more particularly dry skin associated with a low sebum level in particular.

In consequence, the present invention provides the use of medium chain glyceride(s) as an active ingredient for the preparation of a composition for oral and/or parenteral absorption and intended to prevent and/or treat dry and/or delicate keratinous substances.

In a further aspect, the present invention provides the use of medium chain glyceride(s) as an active ingredient for the preparation of a composition for oral and/or parenteral absorption and intended to prevent and/or treat dry and/or delicate skin.

The invention also provides the use of medium chain glyceride(s) as an active ingredient for the preparation of a composition for oral and/or parenteral absorption and intended to treat and/or prevent cutaneous disorders associated with defective excretion and/or secretion of sebum.

In a still further aspect, the present invention also provides the use of medium chain glyceride(s) as an active ingredient for the preparation of a composition for oral and/or parenteral absorption and intended to treat dry hypo-seborrheic skin.

In a still further aspect, the present invention provides the use of medium chain glyceride(s) as an active ingredient for the preparation of a composition for oral and/or parenteral absorption and intended to stimulate sebogenesis.

The present invention also provides the use of medium chain glyceride(s) as an active ingredient for the preparation of a composition for oral and/or parenteral absorption and intended to prevent and/or treat itching and/or tightness.

In a yet still further aspect, the present invention provides the use of medium chain glyceride(s) as an active ingredient for the preparation of a composition for oral and/or parenteral absorption and intended to prevent and/or treat dry or delicate keratinous fibers and/or functional disorders of the pilo-sebaceous unit.

In a yet still further aspect, the present invention provides the use of medium chain glyceride(s) as an active ingredient for the preparation of a composition for oral and/or parenteral absorption and intended to prevent and/or reduce wrinkles associated with cutaneous dryness.

The term “keratinous fibers” means human or animal hair, bristles, and lashes.

The compositions of the invention may be in the form of a food supplement, a nutritional composition, or in the form of a functional food aimed at treating keratinous substances and in particular the skin, and intended for humans or animals.

They may also be in the form of a composition of therapeutic nature.

In a further aspect, the present invention also envisages the use, therapeutic or otherwise, of medium chain glyceride(s), as an active ingredient to prevent and/or treat dry and/or delicate keratinous substances, in particular dry and/or delicate skin, and/or to prevent and/or treat itching and/or tightness, and/or to treat and/or prevent cutaneous disorders and/or disorders of the pilo-sebaceous unit associated with a defect in sebum excretion and/or secretion, and/or to treat hypo-seborrheic dry skin and/or to stimulate sebogenesis and/or to prevent and/or treat dry or delicate keratinous fibers and/or to prevent and/or reduce wrinkles associated with cutaneous dryness.

More particularly, the invention is implemented by oral and/or parenteral absorption.

In a further aspect, the present invention provides a composition for oral absorption and intended to prevent and/or treat dry or delicate keratinous substances, in particular dry and/or delicate skin, characterized in that it comprises, as an active ingredient, at least one medium chain glyceride and at least one other agent that is active in the skin.

The present invention also provides a method or process for preventing and/or treating keratinous fiber and/or substance disorders, in particular as specified above, and/or for stimulating sebogenesis, comprising administering at least an effective quantity of medium chain glyceride(s).

Thus, the present invention also provides a therapeutic or non-therapeutic treatment method to prevent and/or treat dry and/or delicate keratinous substances, in particular dry and/or delicate skin, and/or to treat dry hypo-seborrheic skin and/or to prevent and/or treat itching and/or tightness, and/or to stimulate sebogenesis, and/or to treat and/or prevent cutaneous disorders and/or disorders of the pilo-sebaceous unit associated with a defect in sebum excretion and/or secretion, and/or to treat and/or prevent dry or delicate keratinous fibers and/or to prevent and/or reduce wrinkles associated with cutaneous dryness, comprising administering at least an effective quantity of medium chain glyceride(s).

Administration may in particular be carried out orally or parenterally, and is more particularly carried out orally.

### Medium Chain Glycerides

The term “glycerides” generally encompasses monoglycerides, diglycerides, triglycerides, and mixtures thereof.

In a preferred implementation of the invention, they are more particularly medium chain triglycerides, if appropriate as a mixture with monoglycerides and/or diglycerides.

The medium chain glycerides used in the context of the present invention are generally in the form of mixtures.

Depending on their degree of esterification, they are composed of one or more molecules of medium chain fatty acids, i.e. fatty acids containing 6 to 12 carbon atoms, in particular 8 to 10, as opposed to a long chain fatty acid that has more than 14 carbon atoms and a short chain fatty acid that has 4 to 6 carbon atoms.

The medium chain fatty acids may be constituted by linear or branched, saturated or unsaturated chains that may include one more unsaturated bonds.

More particularly, the fatty acids of the invention have linear and saturated chains.

Thus, the medium chain glycerides are generally composed of medium chain fatty acids extending from caproic acid (C₆) to lauric acid (C₁₂).

The glycerides considered in the context of the invention may be composed of a unique type of fatty acid, especially C₆, C₈, C₁₀ or C₁₂ or, with diglycerides and triglycerides, derived from glycerol esterification by fatty acid molecules with different chain lengths.

More precisely, the medium chain glycerides and in particular medium chain triglycerides of the present invention are composed of 40% to 90%, in particular 45% to 75%, preferably 50% to 70% by weight of C₈ fatty acids. They also have 15% to 60%, in particular 30% to 50% of C₁₀ fatty acids.

In general, they may be in the form of a mixture including a maximum of 2% by weight of C₆ fatty acids.

Similarly, mixtures of medium chain glycerides of the present invention generally have less than 3% and in particular at most 1% by weight of C₁₂ fatty acids.

Example 1 below proposes a composition of a mixture of medium chain triglycerides of the invention.

In general, the medium chain triglycerides used in the present invention are used in the form of mixtures of a plurality of medium chain triglycerides in which the respective proportions may vary depending on the mixtures.

Such mixtures are generally obtained at the end of their fabrication process.

Generally, the medium chain triglycerides are prepared by esterifying glycerol with medium chain fatty acids derived from oils with a high lauric acid content. Coconut and palm nut oils, which respectively contain about 13% and about 8% of C₈/C₁₀ fatty acids, or cuphea oils which, depending on their variety, may contain up to 87% of C₈ (for cuphea painteri) and 81% of C₁₀ (for cuphea carthagenensis), are among the natural sources that are the richest and the most abundant in medium chain fatty acids. Conventionally, these oils are hydrolyzed to liberate their fatty acids from the glycerol, then these fatty acids are separated by fractional distillation. The head fraction of the fatty acids contains medium chain fatty acids. Fractions of C₈ and C₁₀ acids containing only small quantities of C₆ and C₁₂ acids may thus be isolated. An esterification reaction between glycerol and medium chain fatty acids, thus isolated, can produce mixtures of medium chain triglycerides. Different proportions of each of these acids may be obtained to produce different products. Substances containing mainly either the C₈ form or the C₁₀ form, as well as mixtures of these two forms, are commercially available.

These medium chain triglycerides are still routinely known as “captrin”, glyceryl tri(caprylate/capra) or capric/caprylic triglyceride.

Illustrative examples of substances based on triglycerides that are suitable for the invention, and that may be mentioned in particular are those sold under the trade name NEOBEE® M-5 from STEPAN COMPANY OF MAYWOOD, under the trade name CAPTEX® from ABITEC COMPANY, or Delios® V from COGNIS.

The glycerides may also be used in a non-isolated form. They may then be present in the compositions of the invention in the form of at least one vegetable oil or a mixture of vegetable oils containing them and in particular rich in medium chain C₈ to C₁₀ fatty acids.

As an example, they may be cocoa, cuphea, palm nut, and babassu oils.

In this implementation, the quantity of oil is adjusted to produce a quantity of glycerides, and more particularly MCTs, sufficient to obtain the expected effect.

Furthermore, the glycerides may be used in a form which is pure or associated with other compounds in a composition for the utilizations considered in accordance with the invention.

Within the context of the invention, the expression “effective quantity” means the minimum quantity necessary for the expected effect to be observed.

Such a quantity may readily be determined by the skilled person.

In the composition, the glycerides, and more particularly the MCTs, may represent 0.1% to 100% by weight, in particular 15% to 80% and especially 30% to 70% by weight relative to the total composition weight.

Regardless of the variation under consideration, the glycerides and more particularly the MCTs are generally administered in daily doses of 0.5 mg/day to 100 g/day, in particular 1 mg/day to 10 g/day, especially 5 mg/day to 2.5 g/day.

As opposed to the more conventional administration route, namely the topical route, the oral route has the advantage of being easy and fast to use, and also of being generally effective on all keratinous substances.

In one embodiment, a composition of the invention includes less than 2% by weight of petroselinic acid relative to the total composition weight, or even is free of petroselinic acid.

In one embodiment, a composition of the invention may include less than 2% by weight of Vaseline oil relative to the total composition weight, or even be free of Vaseline oil.

In one embodiment, a composition of the invention may be free of active UV filter type agent.

In one embodiment, a composition of the invention may be free of a co-enzyme Q.

In one embodiment, a composition of the invention may be free of a preservative agent from the paraben class (for example methylparaben or propylparaben).

In one embodiment, a composition of the invention may be free of chromium or a chromium salt (chromium (III) chloride, 6 H2O).

In one embodiment, a composition of the invention may be free of sodium caseinate hydrolysate.

When the keratinous substances considered in the context of the invention are human or animal keratinous fibers, like hair, bristles and/or lashes, medium chain glycerides are particularly advantageous to prevent and/or treat the signs of delicateness, such as dryness which is generally evidenced by the brittle aspect of the fiber.

The medium chain glycerides can endow keratinous fibers, in particular human hair or animal fur, with a lustrous appearance.

In particular with dry skin, medium chain glycerides 5 are particularly effective in preventing and/or treating dry skin or delicate skin, in particular to physiologically restore a suitable degree of hydration to the stratum corneum.

In the context of the invention, delicate skin is a skin affected by a deficit in the barrier function, in particular characterized, or susceptible of being characterized, by a deficiency of the constituent lipids of the barrier and/or hydrolipid film. However, a delicate skin does not exhibit, or at least does not exhibit to the same extent, the characteristic features of dry skin, namely itching and tightness.

Further, a delicate skin is different from an allergic skin and also from a “sensitive” skin. Indeed, its reactivity does not arise from an immunological process as with allergic skin, nor from a neurogenic process as with “sensitive” skin.

Thus, the present invention is aimed at dry and/or delicate keratinous substances distinct from allergic or sensitive skin, whether dry or not.

The dryness that can be treated using the invention may be acquired and transitory dryness, i.e. dryness associated with dehydration of the skin, for example by cold, heat, detergents, hard water and/or chemicals such as solvents.

It may also apply to acquired and permanent dryness such as, for example, that due to physiological factors such as chronological ageing of the skin, generally associated with a loss of functionality of the sebaceous glands and thus to a greater or lesser lack of sebum.

Said dryness may also have been acquired as a result of a therapeutic treatment using compounds such as dermocorticoids, retinoids, or puvatherapeutic, or radiotherapeutic type treatments.

Finally, said dryness may be constitutional, i.e. manifested chronically by the subject, or may be of genetic origin, as in ichthyosis, atopia, psoriasis, and hyperkeratosis.

In so far as the inventors have also demonstrated a stimulating action of medium chain glycerides regarding sebogenesis, these compounds have also proved to be advantageous in treating all disorders associated with hypo-seborrhea.

As a consequence, the medium chain glycerides and the compositions of the invention may effectively be used to treat skin exhibiting insufficient secretion and/or excretion of sebum, and disorders generally associated with this type of deregulation such as, for example, a desquamation defect and/or micro-inflammatory dermatitis type manifestations.

The compositions of the invention may be in any of the galenical forms normally used for the mode of administration concerned.

For oral ingestion, numerous forms of oral compositions are possible, especially compositions that may be or may not be medicating, in particular food or nutritional supplements. They are formulated using the usual methods to produce dragees, gelules, gels, emulsions, which may be dry or liquid, tablets, capsules, or solutions.

The medium chain glycerides may also be incorporated into food matrices to produce functional foods such as food bars, enriched foods such as oils, butters, margarines, compressed powders, fibers, gums, chewing gum, dried fruit and vegetables, cereals, chips, pastries, cookies, crackers or as an emulsion in beverages.

When the compositions are more particularly intended for administration to animals, these compositions may be in the form of a food composition of the nutritional support type or as a drug. Regardless of the composition type, it should be formulated in a galenical form appropriate to its administration to the animal race concerned.

The compositions of the invention may be formulated with the usual excipients and components for such oral compositions, namely in particular fatty and/or aqueous components, moisturizing agents, thickeners, preservatives, texturizing agents, flavoring agents and/or coating agents, antioxidants, preservatives, and colorants usual to the food industry.

The formulation agents and excipients for oral composition and in particular for food supplements are known in the art and are not described in detail herein.

In the compositions of the invention, the medium chain glyceride component in the form of a single compound or a mixture may also be associated with an effective quantity of at least one other agent that is active on keratinous substances.

As an example, this other active agent may induce an unwanted effect, such as the appearance of dry skin, especially due to limited sebum production. Examples of such compounds that may be mentioned are corticoids, in particular cortisone, hydrocortisone and betamethasone; indometacine; and retinoic acid derivatives.

The active ingredients that may be associated with the medium chain triglycerides of the invention also include ingredients that are routinely used and/or allowed in the galenical forms intended for oral administration.

These ingredients may be selected from vitamins, minerals, essential lipids and their derivatives, in particular conjugated linoleic acid (or CLA), caprenin (triglyceride of capric, caprylic and behenic acids), oligoelements, polyphenols, flavonoids, phyto-estrogens, antioxidants such as lipoic acid and coenzyme Q10, carotenoids, probiotics, in particular yeasts, prebiotics, proteins and amino acids, mono- and polysaccharides, amino-sugars, phytosterols and triterpene alcohols of vegetable origin.

In particular, they are vitamins A, C, D, E, PP and group B vitamins. Preferred carotenoids are beta-carotene, lycopene, lutein, zeazanthin, and astaxanthin. Particular minerals and oligo-elements that are used are zinc, calcium, magnesium, copper, iron, iodine, manganese, selenium, and chromium (III). Particular polyphenols which may be used are grape, tea, olive, cocoa, coffee, apple, huckleberry, elder, strawberry, cranberry, and onion polyphenols. Preferred phytoestrogens are isoflavones in the free or glycosylated form such as genistein, daidzein, glyciteine, or lignans, in particular those from flax and from schizandra chinensis. The probiotics are preferably selected from the group constituted by lactobacillae and bifidobacteria. A probiotic which is suitable for the invention may also be selected from yeasts. The amino acids or peptides and the proteins containing them, such as taurine, threonine, cysteine, tryptophane, methionine. Preferred lipids belong to the group of oils containing mono- and polyunsaturated fatty acids such as oleic, linoleic, alpha-linolenic, gamma-linolenic, stearidonic acids, long chain omega-3 fatty acids from fish such as EPA and DHA, conjugated fatty acids from vegetables or animals such as CLA (conjugated linoleic acid).

In a particular implementation of the invention, these ingredients may be selected from conjugated linoleic acid (CLA), yeasts, caprenin, polyphenols, flavonoids, carotenoids other than beta-carotene, phyto-estrogens, lipoic acid and coenzyme Q10, probiotics and in particular yeasts, amino-sugars, phytosterols and triterpenic alcohols of vegetable origin. The present invention also provides a treatment method or process comprising the use of the medium chain glyceride(s) of the invention.

A therapeutic or non-therapeutic treatment method of the invention may comprise a single administration.

In a further implementation, administration is repeated, for example 2 to 3 times a day over a day or more and generally for a prolonged period of at least 4 weeks, or even 4 to 15 weeks, if necessary with one or more interruptions.

It is also possible to envisage that the method involves joint administration of another product intended to have a beneficial effect on the keratinous substance under consideration, and in particular comprising at least one agent known to be effective in preventing and/or treating keratinous substance dryness. In particular, it may be an active ingredient as described above. This other product may be administered orally or by a distinct pathway, for example topically.

Similarly, the invention extends to a method involving joint oral administration of medium chain glycerides of the invention and topical or parenteral administration of identical or different medium chain glycerides. This topical administration may be carried out by application to the skin of the treated human or animal in any conventional galenical form, such as a cream, milk, spray, body gel, for example, containing an effective quantity of medium chain glyceride(s).

The examples below are given by way of non-limiting illustration of the field of the invention.

### EXAMPLE 1

### Different Oral Administration Formulation Modes

In this example, the MCTs were sold by COGNIS, under the trade name Delios® V. Their principal physicochemical characteristics were as follows:

| Physicochemical composition | Specifications by weight |
|---|---|
| Moisture | 0.1% maximum |
| Fatty acid distribution | |
| C₆: 0 | 2% maximum |
| C₈: 0 | 54-64% |
| C₁₀: 0 | 34-46% |
| C₁₂: 0 | 1% maximum |
| Acid index (mg KOH/g) | 0.1 maximum |
| Saponification index (mg | 330-345 |
| KOH/g) | |
| Hydroxyl index (mg | 5.0 maximum |
| KOH/g) | |
| Iodine index | 0.5 maximum |

Soft capsule form

| Ingredient/additive | Dose (mg/capsule) |
|---|---|
| MCT | 65 |
| Olive oil | 25 |
| Vitamin E | 5 |

It is possible to consume 3 to 6 capsules per day.

| Ingredient/additive | Dose (mg/capsule) |
|---|---|
| MCT | 45 |
| Fish oil | 30 |
| Blackcurrant seed oil | 10 |
| Vitamin E | 5 |

It is possible to consume 3 to 6 capsules per day.
Blister gelule

| Ingredient/additive | Dose (mg/capsule) |
|---|---|
| MCT | 50 |
| Tristearate | 70 |
| Vitamin E succinate | 5 |
| Colloidal silica | 1 |
| Aerosil ® | |

It is possible to consume 3 to 6 capsules per day.
Single dose gel

| Ingredient/additive | % by weight |
|---|---|
| MCT | 8 |
| Borage oil | 4 |
| Sugar syrup | 30 |
| Maltodextrin | 20 |
| Xanthan gum | 0.7 |
| Soya lecithin | 0.5 |
| Sodium benzoate | 0.2 |
| water | qsp 100 |

It is possible to consume 100 mL to 400 mL per day.

### EXAMPLE 2

A clinical study was carried out on 80 women with winter xerosis between the months of December and March to demonstrate the beneficial effects of a composition in accordance with the invention.

The test composition corresponded to medium chain triglycerides (MCT) packed into soft capsules like those proposed in Example 1.

The daily dose of MCT was equivalent to 2160 mg/day and was administered in the form of 4 capsules per day, taken two in the morning and two in the evening.

The principal criteria for including the subjects in the study were as follows:
age: 50 to 80 years (taking or not taking hormone replacement therapy);
clinical cutaneous dryness score: 1.5 to 3; and
measurement of hydration using CMU125® corneometer (apparatus and method from Courage and Khazaka): 25 and 45 arbitrary units.

The clinical dryness score was produced in accordance with a scale which assessed the dryness intensity on the outside of the leg using the following convention, employing a scale of 0 to 4.5.
score=0/normal skin: regular cutaneous relief. Smooth appearance;
score=0.5/intermediate stage between 0 and 1: slightly irregular cutaneous relief. Slightly rough appearance;
score=1/dehydrated skin: striated cutaneous relief. Slight roughness;
score=1.5/intermediate stage between 1 and 2: more marked striated appearance and rougher;
score=2/dry skin: striated cutaneous relief and a few scales. Rough appearance;
score=2.5/intermediate stage between 2 and 3: presence of scales without flaking, very rough;
score=3/very dry skin: numerous scales and a few flakes. Rough appearance;
score=3.5/intermediate stage between 3 and 4: presence of flakes and numerous scales. Rough appearance;
score=4/extremely dry skin: very many flakes. Very rough appearance;
score=4.5/stage over 4: pathological.

Throughout the study, patients were requested not to change their eating habits, to take no other food supplements, and not to apply any cosmetic compositions to the zones being studied.

The treatment period was 12 weeks. During this study, the following investigations were carried out:
clinical investigations on all of the subjects (40);
biochemical and instrumental investigations for half of the group (20 volunteers living in a community with the same hygiene standards to guarantee homogeneous inclusion criteria for all subjects); and
all 40 subjects filling out a self-evaluation questionnaire.

A statistical analysis of this study was carried out for each investigative criterion by comparing the change in the parameters between D0 and D84.

The investigations were respectively carried out using the following criteria:

a) Clinical Investigations
clinical examination by a dermatologist. This examination in particular evaluated cutaneous dryness and roughness in the face, forearms, hands and legs (4 point score).

b) Instrumental Investigations
corneometry on the leg (inclusion criterion);
sebumeter: measurement of sebum on the forehead and
study of wettability on the forearm.

The wettability technique used has been described by P Humbert et al. (“A New Method To Measure In Vivo Human Skin Hydrophoby”. Int. J. Cosmet. Sci., 2001; 23:347-352 and “Skin Critical Surface Tension: A Way to Assess The Skin Wettability Qualitatively”. Skin Res. Technol., 1996; 2: 91-96).

c) Biochemical Investigations
by taking cotton samples from the leg to measure the lipids present in the stratum corneum, the lipids of the intercorneocytary cement and the sebum (squalene, triglycerides, glycerol, fatty acids, cholesterol).

The results obtained at the end of these investigations are discussed below.

### Results of Clinical Investigations

After taking a composition of the invention for 3 months, the results obtained show a very significant reduction in cutaneous dryness by an average of at least 20% on the most exposed zones and thus the most sensitive to this problem (legs, face, arms, and forearms).

### Results of Instrumental Investigations

The results also bear witness to a very significant increase in the amount of sebum, by more than 35% after taking a composition of the invention for 3 months.

This increase is in agreement with the positive results on hydration and cutaneous wettability. Indeed, an increase in sebum participates in restoring the hydrolipid film and has a positive incidence on hydration. Finally, the sebum production reflects a favorable change in the surface hydrophobicity of the stratum corneum.

### Results of Biochemical Investigations

The data acquired also show that after taking a composition of the invention for 3 months, a very significant increase in the amount of squalene was observed, in particular by at least 75%. This result is in complete agreement with the increase in the amount of sebum shown by the above instrumental investigation.

It should also be noted that oral administration of the composition of the invention encourages the synthesis of lipids of the hydrolipid film such as triglycerides, fatty acids, glycerol, and cholesterol.

In consequence, oral administration of a composition of the invention has a positive effect on restoring the cutaneous hydrolipid film with a very favorable effect on cutaneous dryness.

Further, the 40 volunteers who were questioned regarding the effects of the product reported a significant improvement in cutaneous comfort between D0 and D84 (improvement in softness, tightness and skin suppleness).

Finally, no undesirable event which could be blamed on the composition was observed, meaning that tolerance of the nutritional supplement was excellent.

1-31. (canceled)

32. Method for preparing a composition for oral and/or parenteral absorption and intended to prevent and/or treat at least one selected from the group consisting of dry and/or delicate keratinous substances, dry and/or delicate skin, itching and/or tightness, cutaneous disorders associated with defective excretion and/or secretion of sebum, dry hypo-seborrheic skin, dry and/or delicate keratinous fibers, functional disorders of the pilo-sebaceous unit, wrinkles associated with cutaneous dryness and/or to stimulate sebogenesis, comprising a step of preparing medium chain glyceride(s) as an active ingredient with a suitable carrier.

33. Method according to claim 32, wherein the medium chain glycerides are in the form of a mixture of glycerides the fatty acids of which contain 6 to 12 carbon atoms.

34. Method according to claim 33, wherein the fatty acids contain 8 to 10 carbon atoms.

35. Method according to claim 32, wherein the fatty acids composing the glycerides contain linear and saturated chains.

36. Method according to claim 32, wherein the medium chain glycerides are in the form of a mixture comprising 40% to 90%, by weight of C₈ fatty acids.

37. Method according to claim 36, wherein the mixture comprises 45% to 75% by weight of C₈ fatty acids.

38. Method according to claim 37, wherein the mixture comprises 50% to 70% by weight of C₈ fatty acids.

39. Method according to claim 32, wherein the medium chain glycerides are in the form of a mixture comprising 15% to 60% by weight of C₁₀ fatty acids.

40. Method according to claim 39, wherein the mixture comprises 30% to 50% by weight of C₁₀ fatty acids.

41. Method according to claim 32, wherein the glycerides are in the form of a mixture comprising a maximum of 2% by weight of C₆ fatty acids.

42. Method according to claim 32, wherein said glycerides are in the form of a mixture comprising less than 3% of C₁₂ fatty acids.

43. Method according to claim 32, wherein the mixture comprises at most 1% of C₁₂ fatty acids.

44. Method according to claim 32, wherein the medium chain glycerides are selected from the group consisting of medium chain monoglycerides, diglycerides and triglycerides and mixtures thereof.

45. Method according to claim 32, wherein the medium chain glycerides are constituted by or comprise at least medium chain triglycerides.

46. Method according to claim 32, wherein the glycerides are used in the form of an oil or a mixture of vegetable oils containing them.

47. Method according to claim 32, wherein said composition comprises 0.1% to 100% by weight, of medium chain glycerides relative to the total composition weight.

48. Method according to claim 47, wherein said composition comprises 15% to 80% by weight of medium chain glycerides relative to the total composition weight.

49. Method according to claim 48, wherein said composition comprises 30% to 70% by weight of medium chain glycerides relative to the total composition weight.

50. Method according to claim 32, wherein said composition is intended for oral administration.

51. Method according to claim 32, wherein the composition is in the form of a food supplement, a nutritional composition or in the form of a functional food for the treatment of keratinous substances intended for humans or animals.

52. A composition for oral absorption for the prevention and/or treatment of dry and/or sensitized keratinous substances, wherein it comprises, as an active ingredient, at least one medium chain glyceride and at least one other agent that is active on keratinous substances.

53. A composition according to claim 52, wherein said active agent is selected from the group consisting of vitamins, minerals, essential lipids and their derivatives, in particular conjugated linoleic acid, caprenin, oligoelements, polyphenols, flavonoids, phyto-estrogens, antioxidants such as lipoic acid and coenzyme Q10, carotenoids, probiotics, prebiotics, proteins and amino acids, mono- and polysaccharides, amino-sugars, phytosterols and triterpene alcohols of vegetable origin.

54. A composition according to claim 52, wherein the medium chain glycerides are in the form of a mixture of glycerides the fatty acids of which contain 6 to 12 carbon atoms.

55. Method for preparing a non-therapeutic composition to prevent and/or treat dry and/or delicate keratinous substances, and/or to treat and/or prevent cutaneous disorders and/or disorders of the pilo-sebaceous unit associated with a defect in sebum excretion and/or secretion and/or to treat hypo-seborrheic dry skin and/or to prevent and/or treat itching and/or tightness and/or to stimulate sebogenesis and/or to prevent and/or treat dry or delicate keratinous fibers and/or to prevent and/or reduce wrinkles associated with cutaneous dryness, comprising a step of preparing medium chain glycerides as an active ingredient with a suitable carrier.

56. Method according to claim 55, wherein medium chain triglycerides are used.

57. A non-therapeutic treatment method to prevent and/or treat dry and/or delicate keratinous substances, and/or to treat dry hypo-seborrheic skin and/or to prevent and/or treat itching and/or tightness and/or to stimulate sebogenesis and/or to treat and/or prevent cutaneous disorders and/or disorders of the pilo-sebaceous unit associated with a defect in sebum excretion and/or secretion and/or to prevent and/or treat dry or delicate keratinous fibers and/or to prevent and/or reduce wrinkles associated with cutaneous dryness, comprising administering at least an effective quantity of medium chain glyceride(s).

58. A method according to claim 57, wherein medium chain triglyceride(s) are used.

59. A method according to claim 57, wherein the medium chain glycerides are administered in daily doses of 0.5 mg/day to 100 g/day

60. A method according to claim 59, wherein the medium chain glycerides are administered in daily doses of 1 mg/day to 10 g/day.

61. A method according to claim 60, wherein the medium chain glycerides are administered in daily doses of 5 to 2.5 g/day.

62. A method according to claim 55, wherein the medium chain triglycerides are administered orally and/or parenterally.

63. A method according to claim 55, wherein it comprises joint topical administration of medium chain glycerides.
